(19) United States
(12) Patent Application Publication
(10) Pub. No.: US 2004/0015175 A1
(43) Pub. Date: Jan. 22, 2004

(54) Tube of air bag - design on glove, forceps and vacuum use to open the birth canal - labor delivery

(76) Inventor: Bich Van Nguyen, Silver Spring, MD (US)

Correspondence Address: BICH VAN NGUYEN, 613 Pershing Dr, Silver Spring, MD, 20910, US

(21) Appl. No.: 10/196,255

(22) Filed: Jul. 17, 2002

(51) Int. Cl. A61B 17/42

(52) U.S. Cl. 606/121

(57) ABSTRACT

The present invention relates to technique use device of a tube of air in labor. Design a glove's air system tube wrap around the hand. When the hand wears this design idea's glove. And use it inside a vagina at the time baby's delivery. Air's system can apply inside the tube. It wraps around the hand. This idea's tube of air will help to stretch and open birth canal. With the hope for baby can slide out easier.

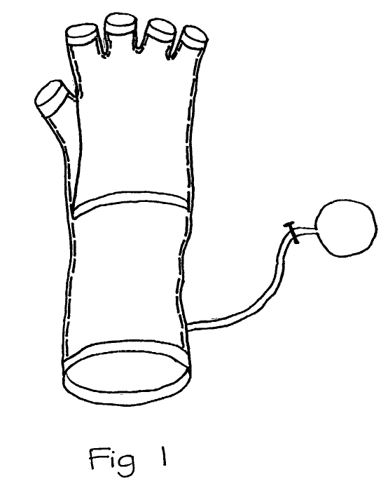

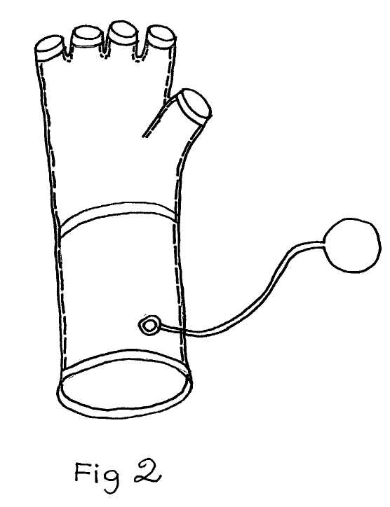

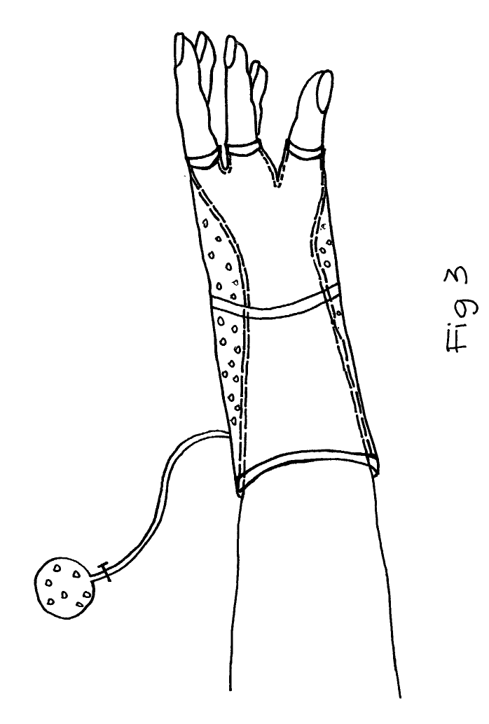

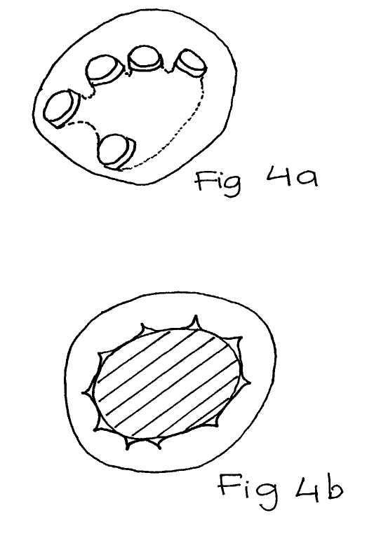

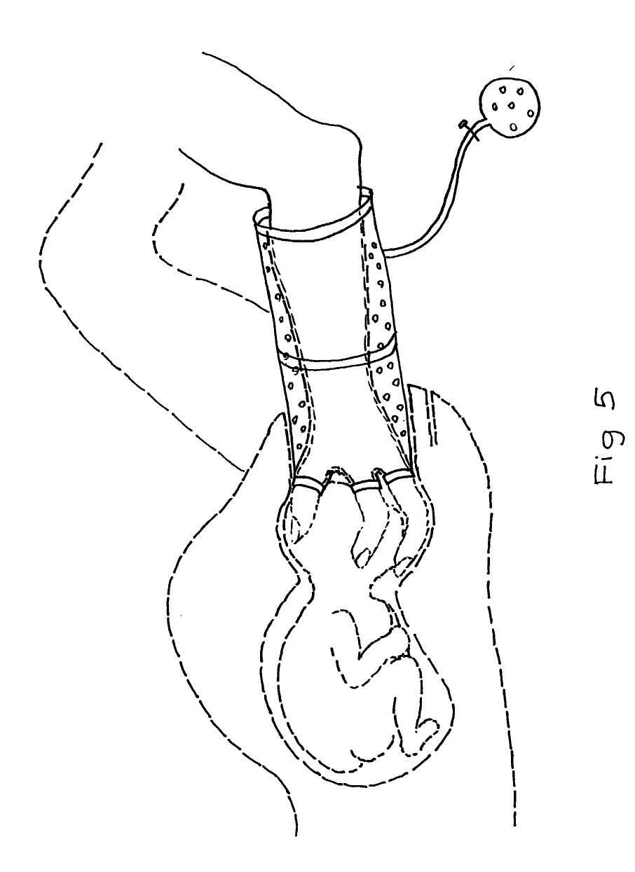

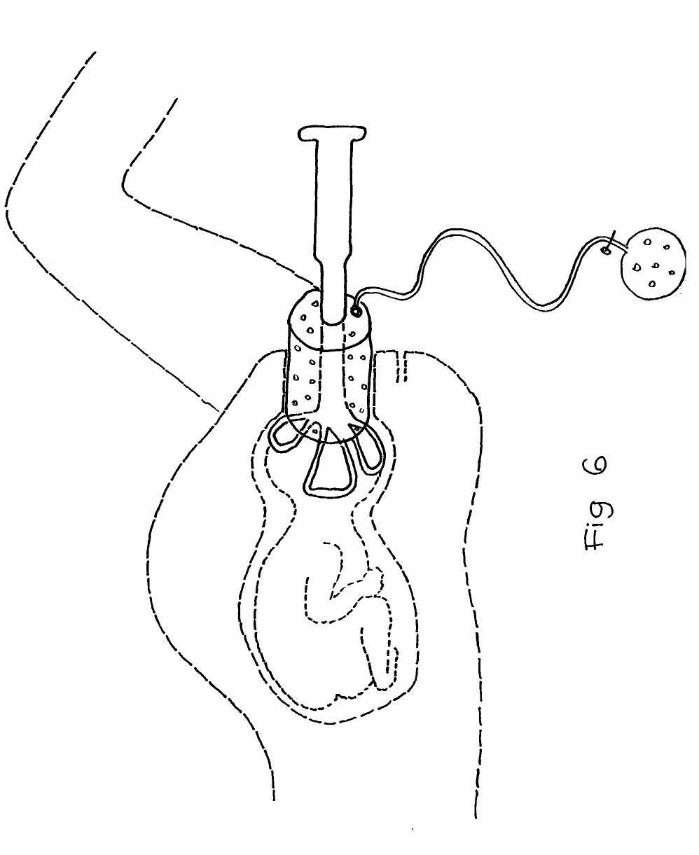

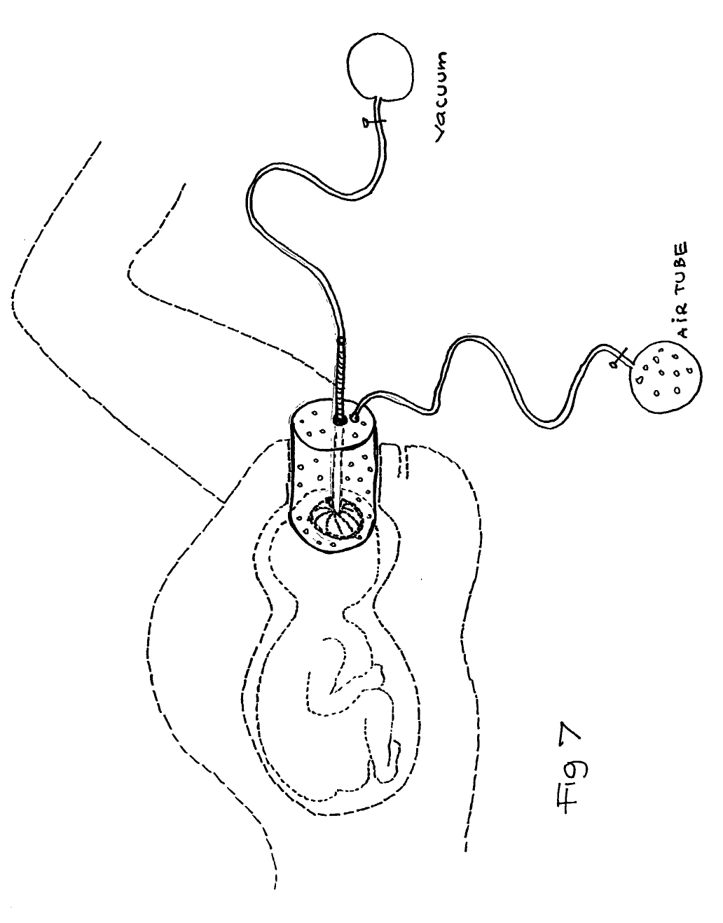

As similar idea, a technique use a tube of air can wrap around the forceps or vacuum. When apply air inside the air's tube, It will stretch and open the birth canal.

The idea tube of air uses for helping to pull the baby out.

### BACKGROUND

1. Field of invention

This invention use for all types of woman in the time of give birth their baby. The baby always has the difficulty time at the time to get out the mother's stomach. A lot of mothers and babies got hurt and damage their health. Sometime these problems cause the lost of their life.

The time of delivery usually too long because the process of delivery depend on the cervix and vagina, The birth canal is so tight or narrow. It wasn't open enough and very difficulty to help the baby get out.

The cervix and vagina or birth canal is the nature way for baby comes out. Birth canal usually is tight and narrow in many case of woman delivery their baby, this cause in many years and the problem for both mother and baby are still looking how to help both.

I was in painful in 20 hours. After tried times used forceps twice and used vacuum three times. My baby finally was out with damage on head cause by forceps and vacuum. She had breathing problems in many months and myself; I never forget this pain in my life.

On the cruise 1999, I was picked to help the show on my vacation cruise. I had to pretend a woman at the time give birth her baby. I dressed up and practiced a few times. This chance reminds me and helped me put my mind work on this idea.

Experience and knowledge I think and found a few ideas. I prayed God help me How and which way is the best and will be easier? The storm came, the wind and waves touch the cruise. I saw many high wave and frightened people on the cruise.

But during that time I was not care much about the storm 0.1 think and see the idea of this device. And my mind worked on it 0.1 went to my room. Draw the idea on papers. I couldn't stay at the same place. The waves from the storm cause the cruise up and down, fall to the left then right.

I worked during the storm; I slide side to side with the waves from the storm. A lot of my friend got seasick. But I think I got an angle touch. Or God blesses. I was not sick or lie down. I felt some thing push or put me to work. Idea is in my mine, It was ready, I hold the pencil and draw. I worried if I wasn't put it down on papers; it might flies away with the wind

More than ten hours, all night I was not rest. I worked and very healthy! When I finished my last drawing. I saw some light out the small window. Then the sun appears. The storm was stop. I look at my work again and the idea I got. I could not believe I did!

I did not make it! God blesses me to do it. And God blesses us out of this pain.

I went up to the top floor. The sun was shinning, It touches my skin. Tear-drop came out. I felt happy, I felt love. Tears run down and down to my face. I could not stop it. A lot of drops of happy and loves.

I looked at the sun. The sun hurt my eyes. I asked . . . Who ever up high there? Is that you? . . . And I kneel down: If you are God or the one led this miracle to me. Please take from me the word Thank you. Please keep me on your arm and bless me always. I am your servant. With all my respected—My hands wipe the tears and It gets so wet, my face wet from all the tears of happy. I hold myself. Dear everybody, another of invention's flower just appears from my brain garden. I kneel and kowtow to the sunshine. Where are you? Are you the one lead me to do all this?

I feel he is in mine and with me of this moment.

The sun shine on me. I see the halo of sunshine. I see a thousand of your diamonds glitter to the surface of water. The sparkling and the reflect from the sun are every where around me.

2. Description of Prior Art

The device consists simply of an air bag wrap around the hand as a special air tube's glove.

When the time baby is ready, This device of an air's glove wear on hand, Then get inside vagina. The air can apply into the air bag up to the size of baby's head or baby's body.

The tube of air wrap around the hand can stretch or make the way wider with the hope: Helps baby can slide out easier.

The birth canal will stretch up to the size of baby's head or baby's body by natural. Why we don't stretch it before? Do we have to wait the baby do it? Or It's better we can stretch it and help. Either way, the birth canal will open. The pain, the time wills the same or less?

Simple and safe, and it is an idea: Wear the glove. Go inside the birth canal. Pump it up or apply the air inside it, Stretch the birth canal make it wider. With the hope helps the baby come out safe, easy and fast.

Some devices are available treat in this field as forceps or vacuum.

(a) The forceps helps but sometime wasn't. It isn't easy because if we hold the baby's head too strong, It can cause damage! It wasn't safe for to pull out the baby.

(b) The vacuum helps but sometime it couldn't help. The vacuum and the baby's head were separate. The suck place where the vacuum attaches a baby's head, It can cause damage for baby or not strong enough to pull the baby out.

(c) The cause of unsuccessful of forceps and vacuum is the narrow way for baby comes out. The muscle of vagina natural is firm and tight. And it still tight on the forceps and vacuum causes problems and couldn't help.

(d) Some time doctor decided secession. It's a lot of work.

### OBJECT AND ADVANTAGE

This invention, a glove's air bag use to help the hand can touch, hold, grab and slide the baby out easier.

The air system wrap around the hand could be use as an excellent device to stretch or make wider the firm of muscle's the vagina canal.

Describe the Air's Glove Bag:

1—the glove made by plastic or strong materials can keep air inside well.

2—The glove has two layers.

a—The first layer is inside, where it touch on the hand and arm.

b—The second layer is attaching to the first layer by two sides: top and bottom. The top: where the place we wear rings on fingers.

c—The bottom: where the end of the glove. It places about ½ on the arm. Two layers press together for it can keep air applied inside.

3—The glove has an air system. A hand pump or electric pump use To apply the air inside the glove. Between two layers the air can applied in full or release.

The air can well keep and hold inside two layers.

The air inside between two layers created a strong tube.

It firm and strong around the arm and hand, accept the fingers.

4—The glove top are not covering the fingers. Two layers press well and attach together as a finger ring on each finger.

5—The glove bottom place about ½ on the arm.

And make it strong for we can hold and pull.

6—The glove's air bag. The press of two layers of glove without cover the finger It look like an air tube wrap around the hand and part of arm.

It can easy make as many kind of float, use for swimmer.

It can be a few sizes small, medium or large.

7—The glove's air bag is well made and reusable or non-reusable.

8—When the glove was apply enough air. The size of the glove will stretch up to the size of the baby size. Or as big as the baby's head size. Then the baby's pressure inside, It wills not got the problem, It will push and pull out easier than before.

It is the safeguard for the baby, because the hand fingers are soft. The fingers have a good connection; also the touch and the feeling of fingers on baby's head are the best help more than others are. It can grab, touch, hold, push up, push down or control the baby head on which way to come out.

Accordingly, several objects and advantage of this invention are:

1) To provide air system tube use for opens wider or stretch vagina canal.

2) To provide the safety for baby by faster's labor.

3) To provide the safety without damages the baby's head.

4) to provide less pain time for the mother

5) To provide a soft device, simple and easy to use.

6) To provide the new technique how to use the air to stretch a vagina canal, that is a simple in design and inexpensive.

7) To provide the firm connection together from the hand, air bag and the baby's head.

8) To provide with the recommendation of position use inside the vagina at the time of labor.

9) To provide with the recommendation of position to help stretch a vagina on time and simple.

10) To provide the size of vagina can stretch by the air system wrap around the hand and arm.

11) To provide to open vagina. The size will get wider or stretch up to the size of the body or baby's head by the air tube wrap around the hand.

12) To provide the easier push out, the release from vagina for baby head can slide out easier.

13) To provide safe for the baby's delivery.

14) To provide the safe and faster for the mother in labor.

15) To provide the birth canal wider.

16) To provide the air tube in a quick use pump or release the air inside the vagina, by use another hand squeeze at the end side air tube of the glove.

17) To provide the air at the end side part, can push up fast into another side It can helps stretch right away or immediately. It pats to open the birth canal.

18) To provide the best technique of strong, firm and safe. The new way to hold the glove, keep, pull this part of glove (the part was out side the birth canal), We can move it slowly out with the baby follow in the hand.

When the baby come out, Baby have to stretch, push and make it wider, So what the different work?

Let's the baby does it? Or

We do it is better?

Use the strong air tube open, stretch and make the birth canal wider!

That the ideas and invention for help both babies

And mother, more safe, less pain and faster!

The same idea but it not a tube of air wrap around the hand, We can design a tube of air system wrap around the forceps or vacuum.

1- A glove's air system design to use in vagina. It uses to stretch and open the birth canal. It uses to help the baby come out easier, safe and faster. It helps faster labor time and less pain for the woman in labor delivery.

2- As similar idea. Apply the tube of air system wrap around the forceps and vacuum. The air tube will stretch and open the birth canal. It wills helps to pull the baby out.
